(19) United States
(12) Patent Application Publication
(10) Pub. No.: US 2015/0376538 A1
(43) Pub. Date: Dec. 31, 2015

(54) FRICTION MODIFIER AND LUBRICATING OIL COMPOSITION

(71) Applicant: JX NIPPON OIL & ENERGY CORPORATION, Tokyo (JP)

(72) Inventors: Yoshitaka MANABE, Tokyo (JP); Kazuo TAGAWA, Tokyo (JP); Ryuichi UENO, Tokyo (JP); Shinji HASEGAWA, Tokyo (JP); Miho FURUKAWA, Tokyo (JP)

(21) Appl. No.: 14/765,643

(22) PCT Filed: Mar. 6, 2014
(86) PCT No.: PCT/JP2014/055863

§ 371 Date: Aug. 4, 2015

(30) Foreign Application Priority Data
Mar. 6, 2013 (JP) 2013-044671

(51) Int. Cl. C10M 133/44; C07D 207/412

(52) U.S. Cl. CPC C10M 133/44; C07D 207/412

(57) ABSTRACT

Provided is a compound which can increase all of the shudder prevention lifetime, transmission characteristic and transmission torque capacity to satisfactory levels when contained in a lubricating oil. Provided are a succinimide compound represented by the following general formula (1), and a friction modifier and a lubricating oil composition containing the succinimide compound.

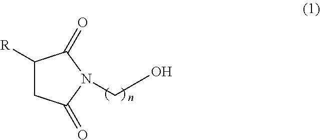

(In the formula (1), R represents a hydrocarbyl group having 8 to 30 carbon atoms; and n represents an integer of 1 to 7.)

### TECHNICAL FIELD

The present invention relates to a new compound which shows a friction modification ability, a friction modifier, and a lubricating oil composition having an improved friction characteristic.

### BACKGROUND ART

Many of automatic transmissions and continuously variable transmissions have torque converters, with which engine torque is transmitted to a transmission through a lubricating oil. A torque converter, due to its structure, cannot transmit power if it does not have a differential rotation between the input side (engine side) and output side (transmission side). However, this differential rotation causes a decrease in power transmission efficiency. Recently, with increasing demands for reducing fuel consumption of vehicles, improvements in power transmission efficiency are required of transmissions more than before. In recent years, it is a frequently adopted method to provide a lock up clutch in a torque converter and directly transmit engine torque to a transmission mechanism in accordance with running conditions, in addition to power transmission via a lubricating oil, so as to improve the power transmission efficiency in a transmission which has a torque converter.

However, when the lock up clutch is activated, torque fluctuations of an engine lead to a less comfortable ride. Therefore, in a conventional lock up mechanism, the lock up clutch is activated only in a high-speed range in which the torque fluctuation of an engine is small, and not activated in a low-speed range. This causes a transmission loss in the torque converter in a low-speed range, for example in starting a vehicle. Therefore, there is still a room for improvements in the conventional lock up clutch, in view of overall fuel consumption.

In order to reduce the transmission loss, a slip control method of activating the lockup clutch even in a low-speed range and absorbing the torque fluctuations of an engine by a relative slip of the clutch, is being introduced these days. However, the slip control of a clutch may cause abnormal vibrations (shudder) originated from a stick slip phenomenon at a friction surface of the lock up clutch, which leads to a less comfortable ride on a vehicle. In order to prevent occurrence of the shudder, it is extremely important to modify a μ-V characteristic so that the friction coefficient (μ) increases along with the increase in the slip speed (V) at the lock up clutch, and such a μ-V characteristic modification ability (shudder prevention performance) as described above is given to a lubricating oil (transmission fluid) (see Patent Literature 1 for example). Since the shudder prevention performance degrades along with the deterioration of lubricating oil, a lubricating oil having a good maintenance ability of shudder prevention performance (shudder prevention lifetime) is required. There is a demand for further improvement of the shudder prevention lifetime, as the row-speed range in which the slip control is carried out is further expanding in pursuing improvement in fuel consumption.

In addition, an automatic transmission or a continuously variable transmission has a wet-type transmission clutch for its transmission mechanism or forward/reverse switching mechanism. A bad friction characteristic of the transmission clutch, for example a too large difference between the dynamic friction coefficient and the static friction coefficient, causes a shock in gear change, which leads to less comfortable ride on a vehicle. Therefore, there is a demand for a lubricating oil for these transmissions which adds a good transmission characteristic to the transmission clutch, in order to reduce shocks which occur when the transmission clutch is engaged.

Making a lubricating oil have the shudder prevention performance and improving the friction characteristic of the transmission clutch are generally carried out by incorporating a friction modifier to the lubricating oil. Therefore, adding a large amount of a friction modifier may be a way to prolong the shudder prevention lifetime of the lubricating oil and improve the transmission characteristic. However, depending on nature of the friction modifier to be incorporated, the friction coefficient (e.g. friction coefficient between metals of a continuously variable transmission) may be largely degraded, which in turn causes another problem of insufficient transmission torque capacity of the transmission. As described above, the shudder prevention lifetime and the transmission characteristic generally have a trade-off relationship with the transmission torque capacity, in an approach of incorporating a friction modifier in a lubricating oil.

### CITATION LIST

### Patent Literatures

Patent Literature 1: JP 2004-155924 A

### SUMMARY OF INVENTION

### Technical Problem

Accordingly, an object of the present invention is to provide a compound which can increase all of the shudder prevention lifetime, transmission characteristic and transmission torque capacity to satisfactory levels when incorporated in a lubricating oil. In addition, the present invention provides a friction modifier which contains the compound, and a lubricating oil composition which contains the friction modifier.

### Solution to Problem

A first aspect of the present invention is a succinimide compound represented by the following general formula (1).

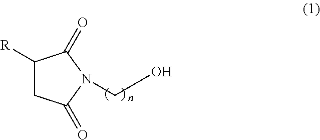

(In the formula (1), R represents a hydrocarbyl group having 8 to 30 carbon atoms; and n represents an integer of 1 to 7).

A second aspect of the present invention is a friction modifier including the succinimide compound according to the first aspect of the present invention.

A third aspect of the present invention is a lubricating oil composition including a lubricant base oil and the friction modifier according to the second aspect of the present invention. The lubricating oil composition of the present invention can be especially preferably used as a continuously variable transmission fluid.

### Advantageous Effect of Invention

According to the succinimide compound of the present invention, by incorporating the succinimide compound in a lubricating oil, it is possible to increase all of the shudder prevention lifetime, transmission characteristic and friction coefficient (transmission torque capacity) of the lubricating oil to satisfactory levels. Therefore, the succinimide compound can be preferably contained as a friction modifier in the lubricating oil composition.

The lubricating oil composition which contains the friction modifier of the present invention has an improved shudder prevention lifetime and a good transmission characteristic, and can also improve the transmission torque capacity since the friction coefficient between metals is also increased. Therefore, the lubricating oil composition can be especially preferably used as an automatic transmission fluid and a continuously variable transmission fluid.

### BRIEF DESCRIPTION OF DRAWINGS

FIG. 1 is an infrared absorption spectrum of a succinimide compound A produced in Production Example 1.

### DESCRIPTION OF EMBODIMENTS

Hereinafter the present invention will be described in detail. It is noted that, unless otherwise mentioned, “A to B” regarding numerical values A and B means “A or more and B or less”. In a case where the unit of the numerical value A is omitted, the unit given to the numerical value B is applied as the unit of the numerical value A.

<1. Succinimide Compound>

### (Structure)

The succinimide compound according to the first aspect of the present invention will be described. The succinimide compound of the present invention has a structure represented by the following general formula (1).

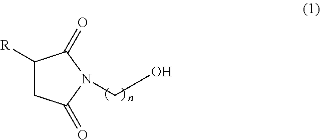

In the above general formula (1), R represents a hydrocarbyl group having 8 to 30 carbon atoms; and n represents an integer of 1 to 7, more preferably an integer of 2 to 4.

Here, specific examples of the hydrocarbyl group having 8 to 30 carbon atoms which can be employed as R include: alkyl groups (which may have a ring structure), alkenyl groups (which may have a ring structure, and whose double bond(s) may be in any position), aryl groups, alkylaryl groups, alkenylaryl groups, arylalkyl groups and arylalkenyl groups.

Examples of the alkyl groups include various linear or branched alkyl groups. Examples of the alkyl groups having ring structure(s) include alkylcycloalkyl groups, cycloalkylalkyl groups, and the like. Examples of the cycloalkyl groups include cycloalkyl groups having 5 to 7 carbon atoms, such as cyclopentyl group, cyclohexyl group and cycloheptyl group. Substitution on the cycloalkyl ring may be in any position.

Examples of the alkenyl groups include various linear or branched alkenyl groups. Examples of the alkenyl groups having ring structures include alkylcycloalkenyl groups, alkenylcycloalkyl groups, cycloalkenylalkyl groups, cycloalkenylalkenyl groups, and the like. The same as above is applied to the cycloalkyl group. Examples of the cycloalkenyl groups include cycloalkyl groups having 5 to 7 carbon atoms, such as cyclopentenyl group, cyclohexenyl group and cycloheptenyl group. Substitution on the cycloalkenyl ring and the cycloalkyl ring may be in any position.

Examples of the aryl groups include phenyl group (having hydrocarbyl substituents), naphthyl group, and the like. In the above alkylaryl groups, alkenylaryl groups, arylalkyl groups and arylalkenyl groups, substitution on the aryl groups may be in any position.

The carbon number of R is 8 to 30, preferably 10 or more, and more preferably 12 or more, and preferably 20 or less. R is preferably an aliphatic hydrocarbyl group (alkyl or alkenyl group which may have a ring structure), and more preferably the aliphatic hydrocarbyl group has a chain moiety. The chain moiety may be a linear chain or a branched chain. However, it is especially preferable that the maximum length of the carbon chain included in the chain moiety (length of the carbon chain from the end closest to the nitrogen atom) is 8 carbons or more (for example, the maximum carbon length included in the chain moiety in 4-(2-ethyldecile)cyclohexyl group (length of the carbon chain from the closest end to the nitrogen atom) is 10 carbons, and the maximum length of the carbon chain included in the chain moiety in 4-(9-cyclopentyldecan-3-yl)cyclohexyl group is 8 carbons).

(Production of Succinimide Compound)

The method of producing the succinimide compound of the present invention is not particularly limited, and the succinimide compound can be produced as follows for example. That is, the succinimide compound can be obtained by a reaction of ω-aminoalkanol and a 2-hydrocarbylsuccinic anhydride (following formula (2)).

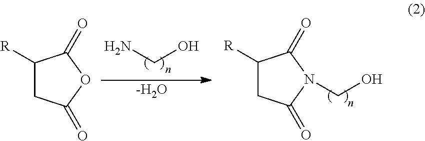

The imidization reaction of the formula (2) can be carried out for example by removing water from the reaction system by azeotropic distillation and phase isolation of water with/from an organic solvent (e.g. o-xylene), by means of a Dean-Stark apparatus under a refluxing condition. Alternatively, after the disappearance of 2-hydrocarbylsuccinic anhydride is confirmed, an imidizing agent (e.g. acetic anhydride and pyridine) can be added to form an imide ring. In the reaction of the formula (2), the hydroxy group of ω-aminoalkanol is not protected. However, the imidization can be carried out with the hydroxy group protected by an appropriate protecting group (e.g. silyl protecting groups such as tert-butyldimethylsilyl group), and thereafter deprotection can be carried out to regenerate the hydroxy group.

If a 2-hydrocarbylsuccinic anhydride having a desired hydrocarbyl group is not commercially available, it can be prepared as follows for example. That is, a 2-hydrocarbylsuccinic anhydride can be prepared by: reacting an organic copper reagent derived from a corresponding hydrocarbyl halide (or a Grignard reagent under existence of a monovalent copper salt) with a tert-butyl haloacetate to introduce a desired hydrocarbyl group into the α-position of a carbonyl group (following formula (3)); further reacting an esterenolate with a tert-butyl haloacetate, to thereby obtain a 2-hydrocarbylsuccinic acid whose carboxy group is protected (following formula (4)); and deprotecting the carboxy group (for example by a treatment with an 85% aqueous solution of phosphoric acid (J. Org. Chem. 2006, 71, 9045-9050), or with trifluoroacetic acid (TFA)) then closing the ring by heating or reaction with acetic anhydride, to thereby obtain a 2-hydrocarbylsuccinic acid hydride (following formula (5)).

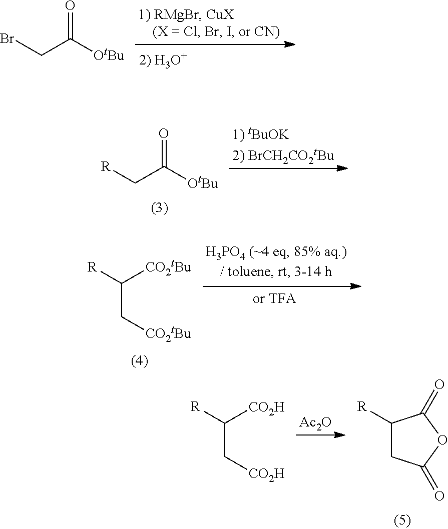

In the succinimide compound of the present invention, it is preferable that the content of a succinic acid half amide compound represented by the following formula (6) or formula (7), which is an impurity formed due to an incomplete imidization in the above formula (2), is small. Specifically, the mass ratio of the total content of the succinic acid half amide impurity represented by the following formula (6) and the succinic acid half amide impurity represented by the following formula (7) to the content of the succinimide compound of the above formula (1) is preferably less than 0.3, more preferably less than 0.2, and particularly preferably less than 0.1. These succinic acid half amide compounds function as free carboxylic acids. Therefore, if their content is too large, corrosiveness to metals may be increased.

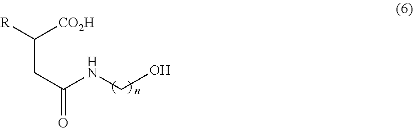

(In the formula (6), R is the same as the R in the formula (1); and n is the same as the n in the formula (1).)

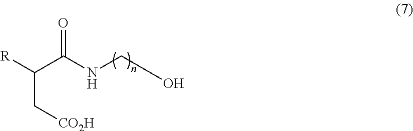

(In the formula (7), R is the same as the R in the formula (1); and n is the same as the n in the formula (1).)

Examples of embodiments of the succinimide compound of the present invention include the following [1] to [13].

[1] an embodiment wherein in the above formula (1), R represents a hydrocarbyl group having 8 to 30 carbon atoms and n represents an integer of 1 to 7;
[2] an embodiment as in [1], wherein n represents an integer of 2 to 4;
[3] an embodiment as in [1], wherein n represents 2;
[4] an embodiment as in any one of [1] to [3], wherein R has 10 or more of carbon atoms;
[5] an embodiment as in any one of [1] to [3], wherein R has 12 or more of carbon atoms;
[6] an embodiment as in any one of [1] to [3], wherein R has 20 or less of carbon atoms;
[7] an embodiment as in anyone of [1] to [6], wherein R represents an aliphatic hydrocarbyl group;
[8] an embodiment as in [7], wherein R has a chain moiety;
[9] an embodiment as in [8], wherein the maximum chain length of the carbon chain included in the chain moiety (which is length of the carbon chain from the end closest to the nitrogen atom) is 8 carbons or more;
[10] an embodiment as in any one of [1] to [6], wherein R represents a linear or branched alkyl group or alkenyl group;
[11] an embodiment as in any one of [1] to [10], wherein the mass ratio of the total content of the succinic acid half amide impurity represented by the above formula (6) and succinic acid half amide impurity represented by the above formula (7) to the content of the succinimide compound represented by the above formula (1) is less than 0.3;
[12] an embodiment as in any one of [1] to [10], wherein the mass ratio of the total content of the succinic acid half amide impurity represented by the above formula (6) and succinic acid half amide impurity represented by the above formula (7) to the content of the succinimide compound represented by the above formula (1) is less than 0.2; and
[13] an embodiment as in any one of [1] to [10], wherein the mass ratio of the total content of the succinic acid half amide impurity represented by the above formula (6) and succinic acid half amide impurity represented by the above formula (7) to the content of the succinimide compound represented by the above formula (1) is less than 0.1.

<2. Friction Modifier>

The friction modifier according to the second aspect of the present invention includes the succinimide compound according to the first aspect of the present invention.

(Content)

The content of the succinimide compound of the present invention in the friction modifier of the present invention is not particularly limited. However, on the basis of the total amount of the friction modifier, the content is preferably 50 weight % or more, more preferably 80 weight % or more, further preferably 90 weight % or more, and may be 100 weight %.

Examples of embodiments of the friction modifier of the present invention include the following [14] to [18].

[14] an embodiment wherein the friction modifier contains the succinimide compound as in any one of the above [1] to [13];
[15] an embodiment wherein the friction modifier contains the succinimide compound as in any one of the above [1] to [13] in an amount of 50 weight % or more on the basis of the total amount of the friction modifier;
[16] an embodiment wherein the friction modifier contains the succinimide compound as in any one of the above [1] to [13] in an amount of 80 weight % or more on the basis of the total amount of the friction modifier;
[17] an embodiment wherein the friction modifier contains the succinimide compound as in any one of the above [1] to [13] in an amount of 90 weight % or more on the basis of the total amount of the friction modifier; and
[18] an embodiment wherein the friction modifier consists of the succinimide compound as in any one of the above [1] to [13].

<3. Lubricating Oil Composition>

The lubricating oil composition according to the third aspect of the present invention includes a base oil and the friction modifier according to the second aspect of the present invention (hereinafter, the friction modifier may be referred to as “polyfunctional succinimide-based friction modifier”).

(Lubricant Base Oil)

A mineral base oil and synthetic base oil that are used for usual lubricating oils can be used as the lubricant base oil in the lubricating oil composition of the present invention, without particular limitations.

Specific examples of the mineral base oil include: oils obtained by refining a lubricating oil fraction produced by vacuum-distilling a topped crude resulting from atmospheric distillation of a crude oil, through one or more treatment (s) such as solvent deasphalting, solvent extraction, hydrocracking, solvent dewaxing and hydrorefining; wax-isomerized mineral oils; and lubricating base oils obtained by isomerizing GTL WAX (gas-to-liquid wax) produced by Fischer-Tropsh process and the like.

Examples of the synthetic lubricating oil include: poly-α-olefins such as 1-octen oligomer and 1-decene oligomer, or hydrogenated products thereof; isobutene oligomers or hydrogenated products thereof; paraffins; alkylbenzenes; alkylnaphthalenes; diesters such as ditridecylglutarate, di-2-ethylhexyladipate, diisodecyladipate, ditridecyladipate and di-2-ethylhexylsebacate; polyolesters such as trimethylolpropanecaprylate, trimethylolpropanepelargonate, pentaerythritol-2-ethylhexanoate, and pentaerythritoipelargonate; polyoxyalkylene glycols; dialkyldiphenyl ethers; and polyphenyl ether. In addition, aromatic synthetic oils such as alkylnaphthalenes, alkylbenzenes and aromatic esters, or a mixture thereof can be given as examples.

In the lubricating oil composition of the present invention, one or more selected from mineral base oils, synthetic base oils, and any mixture thereof can be used as the lubricant base oil. For example, one or more mineral base oils, one or more synthetic base oils, mixed oils of one or more mineral base oils and one or more synthetic base oils and the like, can be given.

The kinematic viscosity, NOACK volatility and viscosity index of the lubricant base oil in the lubricating oil composition of the present invention can be adequately determined, depending on the application of the lubricating oil composition.

(Polyfunctional Succinimide-Based Friction Modifier)

The friction modifier according to the second aspect of the present invention has already been explained. The content thereof is not particularly limited. The content of the succinimide compound represented by the above general formula (1) may be 0.1 to 10 weight % and the like for example, on the basis of the total amount of the lubricating oil composition. Preferable content thereof may depend on the application. For example, in a case where the lubricating oil composition is used for an automatic transmission or a continuously variable transmission, the content is preferably 0.1 weight % or more, and preferably 5 weight % or less.

(Other Additives)

In addition to the above-described lubricant base oil and friction modifier, the lubricating oil composition of the present invention preferably further includes at least one selected from the group consisting of an ashless dispersant, antioxidant, friction modifier other than the succinimide compound of the present invention, anti-wear agent, extreme pressure agent, metallic detergent, viscosity index improver, pour point depressant, corrosion inhibitor, anti-rust agent, metal deactivator, defoamer and coloring agent.

A known ashless dispersant can be used as the ashless dispersant. In a case where an ashless dispersant is contained in the lubricating oil composition of the present invention, the content thereof is, on the basis of the total amount of the lubricating oil composition, that is, on the basis of the total amount of the lubricating oil composition as 100 weight %, normally 0.01 weight % or more, preferably 0.1 weight % or more, and normally 20 weight % or less, and preferably 10 weight % or less.

A known antioxidant can be used as the antioxidant. In a case where an antioxidant is contained in the lubricating oil composition of the present invention, the content thereof is, on the basis of total amount of the lubricating oil composition, normally 5.0 weight % or less, and preferably 3.0 weight % or less, and preferably 0.1 weight % or more, and more preferably 0.5 weight % or more.

A known friction modifier can be used as the friction modifier other than the succinimide compound of the present invention. Examples thereof include oiliness agent-based friction modifiers such as fatty acid esters, and molybdenum-based friction modifiers such as: sulfur-containing molybdenum complexes such as molybdenum dithiocarbamate and molybdenum dithiophosphate; molybdenum complexes not including sulfur, such as molybdenum amine complexes and molybdenum-succinimide complexes; and molybdenum disulfide. In a case where a friction modifier is contained in the lubricating oil composition of the present invention, the content thereof is normally no less than 0.005 weight % and no more than 5 weight %, on the basis of the total amount of the lubricating oil composition.

A known anti-wear agent or extreme pressure agent can be used as the anti-wear agent or the extreme pressure agent. Examples thereof include: phosphorus compounds such as (mono, di, tri-thio) phosphorous or phosphoric esters and zinc dithiophosphates; and sulfur-containing compounds such as disulfides, sulfurized olefins, sulfurized fat and dithiocarbamates. In a case where an anti-wear agent is contained in the lubricating oil composition of the present invention, the content thereof is, on the basis of the total amount of the lubricating oil composition, normally no less than 0.005 weight % and no more than 5 weight %.

A known metallic detergent can be used as the metallic detergent. Examples include alkaline metal sulfonates, alkaline earth metal sulfonates, alkaline metal phenates, alkaline earth metal phenates, alkaline metal salicylates, alkaline earth metal salicylates, and mixture thereof. These metallic detergents may be overbased. In a case where a metallic detergent is contained in the lubricating oil composition of the present invention, the content thereof is not particularly limited. When the lubricating oil composition is for an automatic transmission or a continuously variable transmission, the content thereof is, on the basis of the total amount of the lubricating oil composition, normally no less than 0.01 weight % and no more than 5 weight %, in terms of metal element.

As the viscosity index improver, a known viscosity index improver can be used. Examples thereof include: so-called non-dispersive viscosity index improvers such as polymers or copolymers of one or more selected from various methacrylic acid esters and hydrogenated products thereof; so-called dispersive viscosity index improvers in which various methacrylic acid esters including a nitrogen compound are further copolymered; non-dispersive or dispersive ethylene-α-olefin copolymers and hydrogenated products thereof; polyisobutylenes and hydrogenated products thereof; hydrogenated products of styrene-diene copolymers; styrene-maleic anhydride ester copolymers; polyalkylstyrenes, and the like. As regards the average molecular weight of the viscosity index improver, for example in a case where a dispersive or non-dispersive polymethacrylate is used, its weight-average molecular weight is normally no less than 5,000 and no more than 1,000,000. In a case where a polyisobutylene or a hydrogenated product thereof is used for an internal combustion engine, its number-average molecular weight is normally no less than 800 and no more than 5,000. In a case where an ethylene-α-olefin copolymer or a hydrogenated product thereof is used for an internal combustion engine, its number-average molecular weight is normally no less than 800 and no more than 500,000. In a case where a viscosity index improver is contained in the lubricating oil composition of the present invention, the content thereof is, on the basis of the total amount of the lubricating oil composition, normally no less than 0.1 weight % and no more than 20 weight %.

As the pour point depressant, known pour point depressants such as polymethacrylate polymers can be adequately used, depending on the nature of the lubricant base oil to be used. In a case where a pour point depressant is contained in the lubricating oil composition of the present invention, the content thereof is, on the basis of the total amount of the lubricating oil composition, normally no less than 0.01 weight % and no more than 1 weight %.

As the corrosion inhibitor, known corrosion inhibitors such as benzotriazole-based, tolyltriazole-based, thiadiazole-based and imidazole-based compounds can be used. In a case where a corrosion inhibitor is contained in the lubricating oil composition of the present invention, the content thereof is, on the basis of the total amount of the lubricating oil composition, normally no less than 0.005 weight % and no more than 5 weight %.

As the anti-rust agent, known anti-rust agents such as petroleum sulfonates, alkylbenzene sulfonates, dinonylnaphthalene sulfonate, alkenylsuccinic esters and polyhydric alcohol esters can be used. In a case where an anti-rust agent is contained in the lubricating oil composition of the present invention, the content thereof is, on the basis of the total amount of the lubricating oil composition, normally no less than 0.005 weight % and no more than 5 weight %.

As the metal deactivator, known metal deactivators such as imidazoline, pyrimidine derivatives, alkylthiadiazole, mercaptobenzothiazole, benzotriazole and derivatives thereof, 1,3,4-thiadiazolepolysulfide, 1,3,4-thiadiazolyl-2,5-bisdialkyldithiocarbamate, 2-(alkyldithio)benzimidazole and β-(o-carboxybenzylthio)propionitrile can be used. In a case where a metal deactivator is contained in the lubricating oil composition of the present invention, the content thereof is, on the basis of the total amount of the lubricating oil composition, normally no less than 0.005 weight % and no more than 1 weight %.

As the defoamer, known defoamers such as silicone, fluorosilicone and fluoroalkylether can be used. In a case where a defoamer is contained in the lubricating oil composition of the present invention, the content thereof is, on the basis of the total amount of the lubricating oil composition, normally no less than 0.0005 weight % and no more than 1 weight %.

As the coloring agent, known coloring agents such as azo compounds can be used.

Examples of embodiments of the lubricating oil composition of the present invention include the following [19] to [28].

[19] an embodiment wherein the lubricating oil composition contains a lubricant base oil and the friction modifier as in any one of the above [14] to [18];
[20] an embodiment as in [19], wherein the composition contains the succinimide compound as in any one of the above [1] to [13] in an amount of 0.1 to 10 weight % on the basis of the total amount of the composition;
[21] an embodiment as in [19], wherein the composition contains the succinimide compound as in any one of the above [1] to [13] in an amount of 0.1 to 5 weight % on the basis of the total amount of the composition;
[22] an embodiment as in any one of [19] to [21], wherein the composition further contains at least one selected from the group consisting of an ashless dispersant, antioxidant, friction modifier other than the succinimide compound represented by the above general formula (1), anti-wear agent, extreme pressure agent, metallic detergent, viscosity index improver, pour point depressant, corrosion inhibitor, anti-rust agent, metal deactivator, defoamer and coloring agent;
[23] an embodiment as in any one of [19] to [22], wherein the composition contains an ashless dispersant in an amount of no less than 0.01 weight % and no more than 20 weight % on the basis of the total amount of the composition;
[24] an embodiment as in any one of [19] to [23], wherein the composition contains an antioxidant in an amount of no less than 0.1 weight % and no more than 5.0 weight % on the basis of the total amount of the composition;
[25] an embodiment as in any one of [19] to [24], wherein the composition contains an anti-wear agent or extreme pressure agent in an amount of no less than 0.005 weight % and no more than 5 weight % on the basis of the total amount of the composition;
[26] an embodiment as in any one of [19] to [25], wherein the composition contains a metallic detergent in an amount of no less than 0.01 weight % and no more than 5 weight % on the basis of the total amount of the composition;
[27] an embodiment as in any one of [19] to [26], wherein the composition is used in an automatic transmission; and
[28] an embodiment as in any one of [19] to [26], wherein the composition is used in a continuously variable transmission.

(Application)

By containing the friction modifier (multifunctional succinimide friction modifier) according to the second aspect of the present invention described above, the lubricating oil composition of the present invention has an improved shudder prevention lifetime and a good transmission characteristic, and can also improve the transmission torque capacity since the friction coefficient between metals is also enhanced. Therefore, the lubricating oil composition of the present invention can be especially preferably used as an automatic transmission fluid and a continuously variable transmission fluid.

### EXAMPLES

Hereinafter the present invention will be more specifically described based on Examples and Comparative Examples. However, the present invention is not limited to these Examples.

### Production Examples 1 to 3

Succinimide compounds A to C according to the first aspect of the present invention were produced. IR spectra were measured by means of FT/IR-4100 manufactured by JASCO Corporation. IR spectra of samples which were solid at a room temperature were measured by heating to melt each sample and applying a small amount of the melted sample on a KBr plate. IR spectra of samples in a liquid form at a room temperature were measured by applying a small amount of each sample as it was on a KBr plate.

### Production Example 1

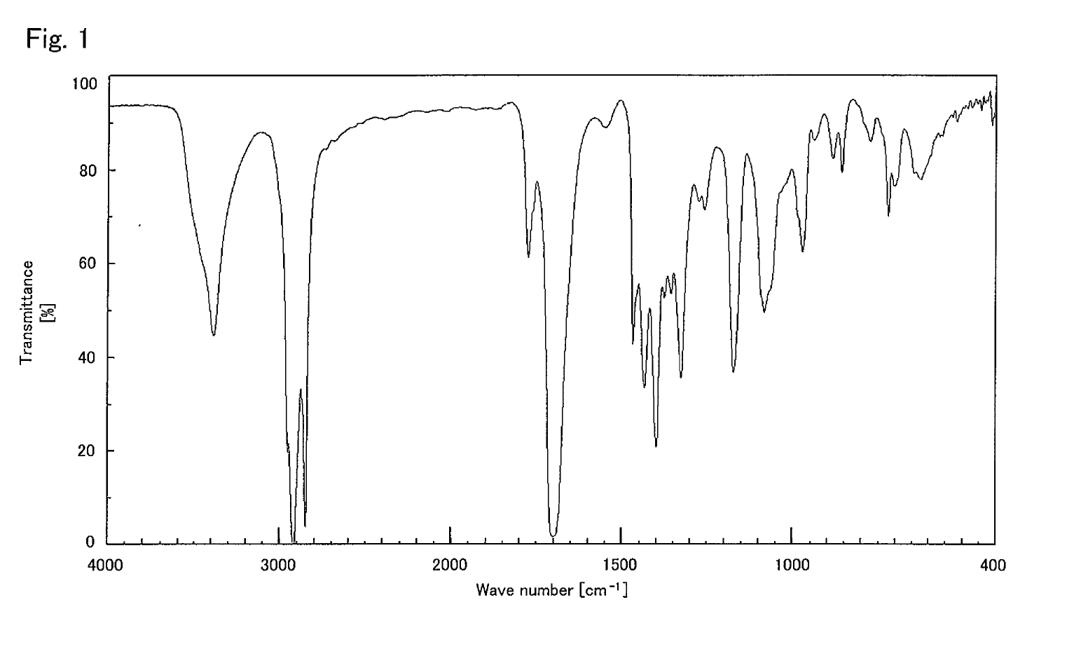

A succinimide compound A of R=octadecenyl group and n=2 in the above general formula (1) was produced by the following procedures. To a 200 mL four-necked flask equipped with a Dean-Stark apparatus, 57.1 mmol (20 g) of octadecenylsuccinic acid anhydride (manufactured by TOKYO CHEMICAL INDUSTRY CO., LTD.), 68.5 mmol (4.2 g) of 2-aminoethanol (manufactured by TOKYO CHEMICAL INDUSTRY CO., LTD.) and 60 mL of o-xylene were added, and an atmosphere inside the flask was substituted with nitrogen. The resultant mixture was heated to reflux, and generated water was removed from the system. After refluxing for three hours, o-xylene and unreacted amine were removed under reduced pressure with small amount of nitrogen flow. The completion of the reaction was confirmed by IR spectrum. About 23 g of the target compound was obtained. The IR spectrum (Neat) of the product is shown in FIG. 1.

### Production Example 2

A succinimide compound B of R=2,4,6,8,10-pentamethyltridecenyl group and n=2 in the above general formula (1) was produced by the following procedures. The same reaction as in the production Example 1 was carried out, except that isooctadecenylsuccinic acid anhydride (manufactured by TOKYO CHEMICAL INDUSTRY CO., LTD.) was used instead of the octadecenylsuccinic acid anhydride of the production example 1. Approximately 23 g of the target compound was obtained.

### Production Example 3

A succinimide compound C of R=an isomeric mixture of linear octadecenyl group (mixture of octadecenyl group isomers having a free valence in different positions in the straight chain of octadecene) and n=2 in the above general formula (2) was produced. The same reaction as in the production example 1 was carried out, except that an isomeric mixture of linear octadecenylsuccinic acid anhydride (manufactured by SEIKO PMC CORPORATION) was used instead of the octadecenylsuccinic acid anhydride used in the production example 1. Approximately 23 g of the target compound was obtained.

### Examples 1 to 3 and Comparative Examples 1 to 3

Lubricating oil compositions according to the third aspect of the present invention (Examples 1 to 3) and lubricating oil compositions for comparison (Comparative Examples 1 to 3) were each prepared to have the composition shown in Table 1. In Table 1, values of ingredient amounts are all based on the total amount of the composition. Regarding units, “wt %” means weight % and “wtppm” means weight ppm.

TABLE 1

| | | | | Comparative | Comparative | Comparative |
|---|---|---|---|---|---|---|
| | Example 1 | Example 2 | Example 3 | Example 1 | Example 2 | Example 3 |
| Base oil (*1) | balance | balance | balance | balance | balance | balance |
| Ashless dispersant (wt %) (*2) | 4.0 | 4.0 | 4.0 | 4.0 | 4.0 | 4.0 |
| Phosphorus-containing anti-wear agent (*3) | 600 | 600 | 600 | 600 | 600 | 600 |
| (wtppm in terms of P) | | | | | | |
| Metallic detergent (*4) | 400 | 400 | 400 | 400 | 400 | 400 |
| (wtppm in terms of Ca) | | | | | | |
| Sulfur-based extreme pressure agent (*5) | 870 | 870 | 870 | 870 | 870 | 870 |
| (wtppm in terms of S) | | | | | | |
| Antioxidant (wt %) (*6) | 0.7 | 0.7 | 0.7 | 0.7 | 0.7 | 0.7 |
| PMA (wt %) (*7) | 17.5 | 17.5 | 17.5 | 17.5 | 17.5 | 17.5 |
| Defoamer (wtppm) | 30 | 30 | 30 | 30 | 30 | 30 |
| Friction modifier (wt %) | | | | | | |
| Compound A (*8) | 1.0 | — | — | — | — | — |
| Compound B (*9) | — | 3.0 | — | — | — | — |
| Compound C (*10) | — | — | 2.0 | — | — | — |
| Glycerol monooleate | — | — | — | — | 1.0 | — |
| Alkyldiethanolamine (*11) | — | — | — | — | — | 1.0 |
| Friction coefficient between metals | 0.123 | 0.131 | 0.115 | 0.137 | 0.116 | 0.108 |
| Shudder prevention lifetime (h) | 186 | 126 | 168 | 0 | 72 | 60 |

(*1) Hydro-cracked mineral oil (kinematic viscosity at 100° C.: 3.3 mm²/s)
(*2) Boron-containing polybutenyl succinimide (N: 2.1 wt %, B: 4.8 wt %)
(*3) Mixture of dialkylphosphite and alkylacidphosphate (P: 16.0 wt %)
(*4) Ca sulfonate (Ca: 11.4 wt %, base number: 300 mgKOH/g)
(*5) Sulfurized olefin (S: 24 wt %)
(*6) Phenol-based antioxidant
(*7) Mixture of two polymethacrylates (Mw: 1.8 × 10⁴ and 4.8 × 10⁴)
(*8) Compound A: R = octadecenyl group, n = 2
(*9) Compound B: R = 2,4,6,8,10-pentamethyltridecenyl group, n = 2
(*10) Compound C: R = isomeric mixture of linear octadecenyl group, n = 2
(*11) Alkyl group: C18 alkyl group

(Evaluation of Friction Coefficient Between Metals)

Friction coefficients between metals were evaluated by means of a block-on-ring friction testing machine (LFW-1, manufactured by FALEX Corporation), under conditions where a lubricating oil composition exist on the friction surface. Testing conditions were: 889 N of load; 0.54 GPa of surface pressure; 0.125 m/s of sliding speed; 80° C. of testing temperature; and 3 minutes of testing time. Test results were evaluated by the average friction coefficient which was an average of the friction coefficient for the time from 1.5 minutes to 2.5 minutes after beginning of the test. Results are together shown in Table 1. If the friction coefficient between metals under the conditions of this test was 0.110 or more, it can be said that the lubricating oil composition can secure a good transmission torque capacity, and the lubricating oil composition is expected to have a good gear change characteristic.

(Evaluation of Shudder Prevention Lifetime)

The shudder prevention lifetime of each of the lubricating oil compositions was evaluated by means of a low speed sliding testing machine specified in JASO M349:2010. The testing method conforms to JASO M349:2010. In the test, μ-V curves were measured at temperatures of 40° C., 80° C. and 120° C. Conforming to JASO M315:2004, the shudder prevention lifetime was determined by: approximating each of the μ-V curves at each measurement temperatures with a quintic function by least square method; differentiating each of the approximate functions at two points of sliding speed (V) of 0.3 m/s and 0.9 m/s, to obtain four gradient values in total; and determining the point of time when any one of the four gradient values at the temperatures of 40° C. and 80° C. became negative as the end of lifetime. Results are shown together in Table 1. If the lifetime is 100 hours or more under the conditions of this test, it can be said that the lubricating oil composition has a good maintenance ability of the shudder prevention performance.

(Evaluation Result)

The lubricating oil compositions of Examples 1 to 3, respectively containing the succinimide compounds A to C of the present invention as friction modifiers, showed good results in both friction coefficients between metals and shudder prevention lifetimes. The lubricating oil composition of Comparative Example 1 containing no friction modifier did not show the shudder prevention performance. The lubricating oil compositions of Comparative Examples 2 and 3, respectively containing glycerolmonooleate and alkyldiethanolamine that were conventional general friction modifiers, showed significantly poor shudder prevention lifetimes. In addition, the lubricating oil composition of Comparative Example 3 did not show a satisfactory friction coefficient between metals.

From the above results, it was shown that the lubricating oil composition containing the succinimide compound of the present invention as a friction modifier can realize both a long shudder prevention lifetime and a good transmission torque capacity because of a high friction coefficient between metals at the same time.

### INDUSTRIAL APPLICABILITY

The lubricating oil composition containing the succinimide compound of the present invention as a friction modifier can be preferably used as a transmission fluid, especially preferably as an automatic transmission fluid and a continuously variable transmission fluid. Specifically, the lubricating oil composition can be preferably used as a lubricating oil for a transmission having a torque converter provided with a lock up clutch, more specifically, a lubricating oil for a transmission having a torque converter which carries out slip control of a lock up clutch.

1. A succinimide compound represented by the following general formula (1): 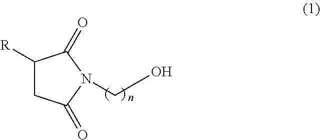
(In the formula (1), R represents a hydrocarbyl group having 8 to 30 carbon atoms; and n represents an integer of 1 to 7).

2. The succinimide compound according to claim 1, wherein a mass ratio of total content of succinic acid half amide impurity represented by the following general formula (6) and succinic acid half amide impurity represented by the following general formula (7) to the content of the succinimide compound is less than 0.3: 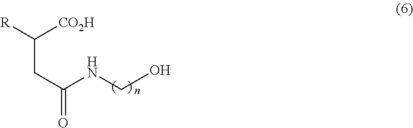
(In the formula (6), R is the same as the R in the formula (1); and n is the same as the n in the formula (1)) 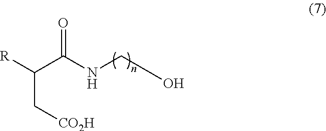
(In the formula (7), R is the same as the R in the formula (1); and n is the same as the n in the formula (1)).

3. A friction modifier comprising the succinimide compound as in claim 1.

4. A lubricating oil composition comprising:
a lubricant base oil; and
a friction modifier as in claim 3.

5. A lubricating oil composition for continuously variable transmissions comprising:
a lubricant base oil; and
a friction modifier as in claim 3.
